(19) United States
(12) Patent Application Publication
(10) Pub. No.: US 2024/0009148 A1
(43) Pub. Date: Jan. 11, 2024

(54) Cystamine Formulations and Uses Thereof

(71) Applicant: Birch Therapeutics, Inc., Novato, CA (US)

(72) Inventor: Artem Zykovich, Novato, CA (US)

(21) Appl. No.: 18/252,873

(22) PCT Filed: Nov. 18, 2021
(86) PCT No.: PCT/US2021/059984

§ 371 Date: May 12, 2023

Related U.S. Application Data
(60) Provisional application No. 63/116,137, filed on Nov. 19, 2020.

(51) Int. Cl. A61K 31/145; A61K 9/28

(52) U.S. Cl. CPC A61K 31/145; A61K 9/28

(57) ABSTRACT

The disclosure provides oral cystamine formulations, methods, and kits useful for treating cystinosis and cysteamine and cystamine sensitive diseases or disorders. The formulations provide a set of varying release compositions that improve the quality of life and offer reduced side-effects.

### CROSS REFERENCE TO RELATED APPLICATIONS

This application claims priority to U.S. Provisional Patent Application No. 63/116,137, filed Nov. 19, 2020, the contents of which are hereby incorporated by reference in their entirety.

### FIELD OF THE INVENTION

The invention relates to compositions of cystamine and treatments for cysteamine and cystamine sensitive diseases or disorders. More specifically, the invention relates to methods, compositions, and kits useful for treating cystinosis and neurodegenerative diseases such as Huntington's, Alzheimer's and Parkinson's disease, and Nonalcoholic Fatty Liver Disease and related diseases.

### BACKGROUND

Cystinosis is a rare, autosomal recessive disorder caused by mutations in the cystinosin gene (CTNS), which encodes a lysosomal cystine transporter (Nat Genet. 1998 April; 18(4):319-24. doi: 10.1038/ng0498-319). Cystine accumulates in lysosomes of affected patients, eventually reaching high concentrations that damage cells. Untreated, patients suffer multi-organ damage, including kidney, spleen, liver, lymph nodes, bone marrow, and eyes. Nephropathic cystinosis is associated with kidney failure that necessitates kidney transplantation. To date, the only FDA approved treatment for nephropathic cystinosis is the sulfhydryl agent, cysteamine. Cysteamine enters the lysosome and converts cystine to cysteine and cysteine-cysteamine mixed disulfide, which can then leave the lysosome through the cysteine and lysine transporters, respectively (Gahl et al., N Engl J Med 347(2):111-21, 2002). Within the cytosol, the mixed disulfide can be reduced by its reaction with glutathione, and released cysteamine can enter the lysosome again to repeat cystine cleavage. Cysteamine can decrease intracellular cystine levels, thereby reducing the rate of progression of kidney failure, and can prevent damage to muscle, thyroid, and other organs (Dohil et al., J. Pediatr 148(6):764-9, 2006).

Cystagon®, an immediate-release formulation of cysteamine bitartrate, was the first FDA approved treatment for cystinosis. The goal of cysteamine therapy is to maintain white blood cell cystine levels below 1 nmol half cystine/mg white cell protein. To maintain the desired level, Cystagon® is commonly administered every six hours, and hence requires interruption of sleep. Such a burdensome dosing regimen deters adherence to the prescribed medication schedule (Levtchenko et al. Pediatric Nephrology 21:110 (2006)).

To address this issue, Raptor Pharmaceuticals developed Procysbi®, an enteric-coated formulation of cysteamine bitartrate, which is released in the small intestine, allowing for twice-daily dosing. However, this new formulation requires the same total daily dose as the immediate-release formulation. Because doses are reduced from four time to two times per day, the unit dose should be double that of Cystagon®. In many patients, the higher dose results in higher peak plasma cysteamine concentrations. Cysteamine can produce body odor and halitosis when ingested in therapeutically effective amounts. Most patients also experience gastrointestinal side effects, including nausea, vomiting, and stomach pain. The halitosis, body odor, and gastrointestinal side effects have all been shown to be associated with high peak cysteamine blood levels (Gangoiti, Br J Clin Pharmacol. 2010 September; 70(3): 376-382). In a clinical trial comparing immediate and delayed formulation regimens, the incidence of gastrointestinal adverse effects was three times higher when patients were treated with Procysbi® compared to the same patients on Cystagon® (Langman et al. Clin. J. Am. Soc. Nephrol . CJN-12321211 (2012)).

Additionally, both FDA approved formulations have stability problems, as cysteamine is oxidized when exposed to the atmosphere and/or high temperatures. The European Medicines Agency Summary of Product Characteristics for Procysbi® (Annex I) specifies that capsules should be used within 30 days after opening the container.

Accordingly, there is a need for improved treatment regimens for cystinosis and other cysteamine or cystamine sensitive diseases that would address the shortcomings of existing therapeutic formulations, including adverse side effects (the consequence of high peak of plasma cysteamine concentrations) and poor cysteamine stability (due to oxidation, which results in a short shelf life).

### SUMMARY OF THE INVENTION

In a first aspect, the present invention provides a composition comprising cystamine or a pharmaceutically acceptable salt thereof and one or more pharmaceutically acceptable carriers, diluents, or excipients.

In a second aspect, the present invention provides a unit dosage form of cystamine or a pharmaceutically acceptable salt thereof comprising one or more of an immediate release (IR) formulation and/or a large intestine release (LIR) formulation of cystamine or a pharmaceutically acceptable salt thereof.

In a third aspect, the present invention provides a method of treating a subject with a cysteamine or cystamine sensitive disease or disorder, comprising administering to the subject a therapeutically effective amount of a composition, formulation, or unit dosage form of cystamine as disclosed herein.

In a fourth aspect, the present invention provides a formulation of cystamine comprising a core comprising cystamine and a binder and one or more time-release coatings surrounding the core, wherein the one or more coatings release the cystamine when the formulation reaches the large intestine, or wherein the formulation has a Cₘₐₓ more than 4 hours when administered therapeutically.

In a fifth aspect, the present invention provides a kit comprising a composition, formulation, or unit dosing form of cystamine as disclosed herein and instructions for the administration of the composition, formulation, or unit dosing form for the treatment of a cysteamine or cystamine sensitive disease or disorder

### BRIEF DESCRIPTION OF THE DRAWINGS

FIG. 1 shows plasma cysteamine concentration-time curve for fasted mice following administration of cysteamine (PO) or cystamine (PO). Error bars represent the standard error of the mean (n=5).

FIG. 2 shows plasma cysteamine concentration-time curve for fasted and fed mice following administration of cysteamine (PO). Error bars represent the standard error of the mean (n=5).

FIG. 3 shows plasma cysteamine concentration-time curve for fasted mice following administration of cysteamine (PO) and fed mice following administration of cystamine (PO). Error bars represent the standard error of the mean (n=5).

FIG. 4 shows plasma cysteamine concentration-time curve for fasted mice following administration of cysteamine (PO) and cysteamine (IC). Error bars represent the standard error of the mean (n=5).

FIG. 5 shows plasma cysteamine concentration-time curve for fasted mice following administration of cysteamine (PO) and cystamine (IC). Error bars represent the standard error of the mean (n=5).

### DETAILED DESCRIPTION

Cystamine, a disulfide that can be reduced to cysteamine in the cytoplasm, decreases lysosomal cystine in cystinosis fibroblast cell culture comparably to cysteamine (Thoene, Intracellular cystine depletion by aminothiols in vitro and in vivo, 1976). Cystamine dihydrochloride is stable to the atmosphere and at temperatures below 200 ° C. Improved formulations and improved dosing regimens of cystamine and cystamine salts, including cystamine dihydrochloride, can avoid high peak of plasma cysteamine concentration, producing sustained and efficacious depletion of lysosomal cystine with reduced side-effects, all with an air- and/or high temperature-stable formulation.

Accordingly, in one aspect, the present invention provides compositions comprising cystamine or a pharmaceutically acceptable salt thereof and one or more pharmaceutically acceptable carriers, diluents, or excipients. The pharmaceutically acceptable salts of cystamine may be any salt suitable for therapeutic administration in an animal. In certain embodiments, the pharmaceutically acceptable salt is cystamine dihydrochloride. Unless otherwise indicated or clear from context, the term “cystamine” as used herein refers to both cystamine and to pharmaceutically acceptable salts thereof.

The compositions of the invention also include and refer to certain formulations and/or unit dosage forms for therapeutic administration. As used herein the terms “formulation” or “formulated for” are used to refer to compositions that include certain components to achieve a desired administration, pharmacokinetic, and/or therapeutic result. In a non-limiting example, “oral formulation” or “formulated for oral administration” or the like refers to a composition of cystamine as disclosed herein that comprises certain components (e.g. excipients, carriers, diluents, etc.) that allow for an oral therapeutic route of administration of the composition. As used herein “unit dosage form” refers to a composition formulated for therapeutic administration, i.e. containing one therapeutic dose of cystamine or a pharmaceutically acceptable salt thereof. Unit dosage forms may include a single formulation or multiple formulations in combination, i.e. they may comprise one or more different compositions of cystamine or a pharmaceutically acceptable salt thereof. Unit dosage forms may also be a single form of the compositions of the invention (e.g. a single tablet or a single injection), or they may comprise multiple forms of the compositions of the invention (e.g. multiple tablets and/or injections), that are to be administered in combination, i.e. they may comprise one or more different forms of the compositions of the invention. As used herein, administration in combination includes concurrent administration but is not so limited, i.e. administration in combination also includes sequential administration, including administration spaced at fixed times apart.

The compositions of the invention may be prepared in any format suitable for administration, including but not limited to tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders. In certain non-limiting embodiments, the compositions may formulated as tablets, mini-tablets, capsules, or as granules and/or a powder.

The compositions described herein generally comprise cystamine and a pharmaceutically acceptable carrier, diluent, or excipient. Such compositions are substantially free of non-pharmaceutically acceptable components, i.e., contain amounts of non-pharmaceutically acceptable components lower than permitted by US regulatory requirements at the time of filing this application. Pharmaceutically acceptable carriers, diluents, or excipients are known to those of skill in the art.

The compositions of the invention may be administered by any suitable route. Routes of administration include, but are not limited to, oral, rectal, vaginal, buccal, transmucosal, topical, transdermal, inhalation, parenteral, intravascular, intraarterial, intravenous, subcutaneous, intracutaneous, intradermal, intramuscular, intraarticular, intrathecal, intracerebral, intraventricular, intraperitoneal, intratracheal, intranasal, sublingual, intraocular, or intratumoral administration. In certain embodiments, the compositions of the invention are formulated for oral administration.

The compositions of the invention may be immediate release (IR) or large intestine release (LIR) formulations, or combinations thereof. As used herein, an “immediate release” formulation, or “formulated for immediate release,” refers to a composition that is developed to dissolve without delay or prolonged dissolution or absorption of the cystamine. Generally, an IR formulation formatted for oral administration is swallowed whole and instantaneously disintegrates to make the drug available for absorption and subsequent pharmacologic action. As used herein “large intestine release” formulation or “formulated for large intestine release” refers to a compositions that is developed to allow for release of a substantial portion of the drug in the large intestine, with a Cₘₐₓ of more than 4 hours after administration of the formulation.

In certain embodiments, the compositions of the invention may be gastro-retentive (GR) formulations, alone or in combination with one or more of IR and/or LIR formulations. As used herein, “gastro-retentive” formulation or “formulated for gastric retention” refers to a composition that is developed to allow for prolonged residence time of the drug in the stomach relative to an immediate release formulation. “Gastro-retentive” as used herein is also used to refer to the administration of an IR formulation with food consumption, which would mimic formulations designed for prolonged residence time of the drug in the stomach without food.

Development of IR, LIR, and GR formulations are within the purview of one of skill in the art, and may be adjusted to provide for specific release profiles. Exemplary formulations for LIR include, but are not limited to, Evonik's EUDRAGIT® RL and RS technology.

The compositions of the invention may include any combination of two or more formulations in any ratio, for example an IR formulation in combination with an LIR and/or a GR formulation.

In certain embodiments of the invention, a unit dosage form comprises one or more doses of a particular formulation, e.g. an IR formulation, together with one or more doses of a different formulation, e.g. a LIR formulation, where the doses are not together within the same composition or formulation. For example, in certain embodiments, an oral unit dosage form comprises one or more tablets of a particular formulation, e.g. an IR formulation, together with one or more tablets of a different formulation, e.g. a LIR formulation.

In a non-limiting example, the present invention includes a formulation comprising a core comprising cystamine and a binder and one or more time-release coatings surrounding the core, wherein the formulation releases the cystamine when it reaches the large intestine, or wherein the formulation has a Cₘₐₓ of more than 4 hours when administered therapeutically. As used herein, and would be understood by one of skill in the art, time-release coatings are coatings that surround the core containing cystamine, and comprise pharmaceutically acceptable carriers, diluents, and/or excipients that provide a desired release profile of cystamine upon administration of the composition. In certain embodiments, the time-release coatings do not contain cystamine. In certain embodiments, the formulations of the present invention include oral formulations comprising a core comprising cystamine and a binder, and 1, 2, 3, 4, or 5 time-release coatings surrounding the core. A single time-release coating may be used, or multiple time-release coatings may be used in combination to provide a desired release profile; the specific selection of a single or combination of time-release coatings and the compositions thereof is within the purview of one of skill in the art.

In certain embodiments, the compositions of the invention comprise a core comprising cystamine formulated for large intestine release, and one or more time-release coatings comprising cystamine formulated for immediate and/or gastro-retentive release.

In certain embodiments, the time-release coatings used with the cystamine formulations of the invention are intermediate coatings. As used herein, “intermediate coatings” refers to coatings that surround the core containing cystamine, that provide one or more desired properties to the formulation such as stability and the like. In certain embodiments, the time-release coatings used with the cystamine formulations of the invention are functional coatings. As used herein, “functional coatings” refers to coatings that impart desired properties onto the cystamine in the formulation, such as a particular release profile, odor or taste masking, etc. In certain embodiments, the cystamine formulations of the invention comprise one or more intermediate coatings and one or more functional coatings.

In certain embodiments, exemplary carriers, diluents, and excipients for the formulation of the IR, LIR, and/or GR compositions of the invention include, but are not limited to, those presented in Table 1. In certain embodiments, the carriers, diluents, or excipients of Table 1 are for use with oral LIR formulations.

TABLE 1
Exemplary Carriers, Diluents, and Excipients

| | |
|---|---|
| Core | Fillers: Microcrystalline cellulose, Lactose Monohydrate, |
| | DCP, Neusilin, Starch, Mannitol, Maltose, Isomalt, |
| | cyclodextrins (α, β, γ substituted cyclodextrines, |
| | polymerized cyclodextrines, any cyclodextrins |
| | (α, β, or γ) or modified α, β, or γ cyclodextrin where |
| | the cyclodextrin is substituted with C₁₋₄ alkyl, |
| | hydroxy-C₁₋₄ alkyl, dihydroxy C₁₋₄ alkyl, CO(C₁₋₄ |
| | alkyl), or any combination thereof, etc.) |
| | Binders: Povidones, HPMC, HPC, PVA's, Sodium alginate, |
| | Natural and artificial polymers with viscosity ranging |
| | from 1 mpa*s to 100,000 mpa*s |
| | Disintegrants: Sodium Starch Glycolate, Crospovidone, |
| | Croscarmellose Sodium, Starch |
| | Lubricants and glidants: Magnesium Stearate, stearic acid, |
| | talc, colloidal silicon dioxide |
| Intermediate | Binders: Povidones, HPMC, HPC, PVA's, Sodium alginate, |
| Coating | Natural and artificial polymers with viscosity ranging |
| | from 1 mpa*s to 100,000 mpa*s |
| | Salts: Succinic Acid, Fumaric Acid, Citric Acid, Glutaric |
| | Acid, Tartaric Acid, Sodium Acetate and other organic |
| | acids |
| Functional | Eudragit ® RL (Ammonio Methacrylate copolymer |
| Coating | Type A, Aminoalkyl Methacrylate copolymer Type A) |
| | Eudragit ® RS (Ammonio Methacrylate copolymer |
| | Type B, Aminooalkyl Methacrylate copolymer Type B) |
| | Odor-masking agents |

The particular uses recited in Table 1 (e.g. core, binder, etc.) are intended to be exemplary and not intended to be limiting. One of skill in the art will readily understand that various formulation components may be used for different purposes and in different ways. Moreover, additional suitable formulation components known to those of skill in the art may be used with the compositions of the invention, whether specifically enumerated above or not, and as if they had been specifically listed herein.

In another aspect, the present invention provides methods of treating a subject with a cysteamine or cystamine sensitive disease or disorder, comprising administering to the subject a therapeutically effective amount of the cystamine compositions, formulations, or unit dosage forms as disclosed herein. And used herein a “cysteamine or cystamine sensitive disease or disorder” is any disease or disorder that would benefit (e.g. result in a reduction or elimination of symptoms or disease/disorder components) from the administration of cysteamine or cystamine to a subject suffering from such a disease or disorder. In certain embodiments of the methods disclosed herein, the compositions, formulations, or unit dosage forms are administered orally.

The present invention overcomes certain shortcomings associated with current therapeutics for the treatment of cysteamine or cystamine sensitive diseases or disorders by maintaining cysteamine or cystamine at a constant level. By “maintaining a constant level” it is meant maintaining a therapeutically effective level in a subject over the majority of a 24 hour period, wherein the level varies by no more than about 50%, by no more than about 40%, by no more than about 30%, by no more than about 25%, by no more than about 20%, by no more than about 15%, by no more than about 10%, by no more than about 5%, or by no more than about 3% of the desired level over time. It will be understood by one of skill in the art that with certain dosing regimens, there may be variances greater than the recited percentages, for example when an IR formulation is administered to the subject. By “over the majority of a 24 hour period” it is meant for at least 60%, at least 70%, at least 75%, at least 80%, or at least 85% of the time over any consecutive 24 hour period.

In certain embodiments, the composition, formulation, or unit dosage form is administered at an amount sufficient to maintain white blood cell cystine levels below 2 nmol half cystine/mg white cell protein. In other embodiments, the composition, formulation, or unit dosage form is administered at an amount sufficient to maintain white blood cell cystine levels below 1 nmol half cystine/mg white cell protein. (For assay information, see, e.g., http://cystinosiscentral.org/Method/wbcproc.html.)

As used herein, the term “subject” is used to refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In certain embodiments, the subject is a human.

In certain embodiments of the methods disclosed herein, the LIR formulations when administered have a Cₘₐₓ of more than 4 hours after administration, of 4.5 or more hours after administration, of 5 or more hours after administration, of 5.5 or more hours after administration, or of 6 or more hours after administration.

In the methods of the invention, the compositions, formulations, or unit dosage forms may be administered by any suitable schedule as could be determined by one of skill in the art, and frequency of administration may vary from individual to individual. In certain embodiments, the composition, formulation, or unit dosage form of the invention may be administered 2 times per day; in other embodiments, the composition, formulation, or unit dosage form may be administered 3 times a day.

In certain embodiments of the methods described herein, the composition, formulation, or unit dosage form of the invention is administered before food, for example between about 0-60 minutes before food, between about 0-45 minutes before food, between about 0-30 minutes before food, or between about 0-15 minutes before food, or about 60 minutes before food, about 45 minutes before food, about 30 minutes before food, about 15 minutes before food, or about 0 minutes before food.

Specific dosing levels may be varied in accordance with the need of the individual subject and the specific composition, formulation, or unit dosage form being administered, and will vary from individual to individual. The dose administered to a subject, in the context of the present invention, should be sufficient to effect a beneficial therapeutic response in the subject over time, to delay onset of disease, or to inhibit disease progression. Thus, the composition is administered to a subject in an amount sufficient to alleviate, reduce, and cure or at least partially delay or arrest symptoms and/or complications from the disease. An amount adequate to accomplish this is defined as a “therapeutically effective dose.” In general, the total daily dose of cystamine may be from about 0.01 g/m² body surface area per day to about 100 g/m² body surface area per day, from^{0.1} g/m² body surface area per day to about 50 g/m² body surface area per day, from^{0.5} g/m² body surface area per day to about 25 g/m² body surface area per day, from^{1.0} g/m² body surface area per day to about 10 g/m² body surface area per day, from^{1.0} g/m² body surface area per day to about 5.0 g/m² body surface area per day, from^{1.0} g/m² body surface area per day to about 3.0 g/m² body surface area per day, or from^{1.0} g/m² body surface area per day to about 2.0 g/m² body surface area per day. In certain embodiments, the total daily dose of cystamine is about 1.35 g/m² body surface area per day or less.

The cysteamine or cystamine sensitive diseases or disorders that may be treated using the methods of the invention include, but are not limited to, inflammatory and fibrotic disorders, neurodegenerative diseases, including Alzheimer's, Huntington's, and Parkinson's diseases, metabolic diseases including diabetes, metabolic syndrome, and the spectrum of fatty liver diseases, including non-alcoholic steatohepatitis (NASH), alcoholic steatohepatitis, and non-alcoholic fatty liver disease (NAFLD). In certain embodiments, the cysteamine or cystamine sensitive disease or disorder is cystinosis. In certain embodiments, the cystinosis is nephropathic cystinosis.

The methods and unit dosage forms as disclosed herein may utilize any combination IR, LIR, and GR formulations in any proportions, even if not specifically enumerated. A suitable combination and ratio of formulations may be derived by one of skill in the art. Similarly, a suitable dosing regimen of these combinations may be derived by one of skill in the art.

In a non-limiting example, the methods of the invention include the administration of ¼ of a daily dose of cystamine to a subject before breakfast, wherein the dose is a combination of equal proportions of IR and GR formulations, ¼ of a daily dose of cystamine to the subject before lunch, wherein the dose is a combination of equal proportions of IR and GR formulations, and ½ of a daily dose of cystamine to the subject before dinner, wherein the dose is a combination of equal proportions of IR, GR, and LIR formulations. In a second non-limiting example, the methods of the invention include the administration of ½ of a daily dose of cystamine to a subject before breakfast, wherein the dose is a combination of equal proportions of IR, GR, and LIR formulations, and ½ of a daily dose of cystamine to the subject before dinner, wherein the dose is a combination of equal proportions of IR, GR, and LIR formulations.

In certain embodiments, the unit dosage forms of the invention comprise one or more IR formulations in combination with one or more LIR formulations. In certain embodiments, the unit dosage forms comprise an IR formulation in combination with an LIR formulation. In certain embodiments, the IR formulation is separate from, but may be administered at the same time as, the LIR formulation. In a non-limiting example, the unit dosage form may include one or more tablets of IR cystamine and one or more tablets of LIR cystamine. Such unit dosage forms may comprise in total from about 10% to about 60% of a daily dose of cystamine, or any range or percent level within this range even if not specifically enumerated, for example 20% to 40%, or 20%, or 40%. In unit dosage forms comprising an IR formulation and an LIR formulation, the IR formulation may comprise from about 10% to about 30% of a daily dosage of cystamine (or any range or percent level within this range even if not specifically enumerated), and the LIR formulation may comprise from about 15% to about 35% (or any range or percent level within this range even if not specifically enumerated) of a daily dosage of cystamine. For example, the IR formulation within such a unit dosage form may comprise about 12% to about 18% of a daily dose of cystamine and the LIR formulation about 20% to about 27% of a daily dose of cystamine. As used herein, “about” means±5% or less of the recited value.

In a non-limiting example, the methods of the invention include the administration of 40% of a daily dose of cystamine to a subject before breakfast, wherein the dose is a combination of 16.6% of IR, and 23.4% of LIR formulations, 40% of a daily dose of cystamine to a subject in about 8 hours after the first dose and before diner, wherein the dose is a combination of 16.6% of IR, and 23.4% of LIR formulations, and 20% of a daily dose of cystamine to the subject before going to bed at night time, wherein the dose consists of LIR formulations. Additional suitable combinations/dosing regiments can be readily derived by one of skill in the art.

In certain embodiments, the compositions, formulations, and unit dosage forms of the invention are substantially free of cysteamine. By “substantially free” it is meant that the compositions comprise less than 10%, less than 5%, less than 4%, less than 3%, less than 2%, less than 1%, or an undetectable amount of cysteamine. In certain embodiments, the GR formulations of the invention are not enterically coated. In certain embodiments, the time to Cₘₐₓ of cystamine of any of the compositions, formulations, and unit dosage forms of the present invention, in particular the IR and LIR formulations, is not between 2-4 hours after administration. In certain embodiments, the compositions, formulations, and unit dosage forms of the present invention do not provide increased delivery of cystamine to the small intestine.

### EXAMPLES

### Example 1: Pharmacokinetic Study of Cystamine Dihydrochloride

Cystamine dihydrochloride and cysteamine hydrochloride were administered to rats 1) orally on an empty stomach; 2) orally on full stomach; and 3) intra-cecum to evaluate the pharmacokinetics of cystamine and to compare it to cysteamine pharmacokinetics. Oral administration (PO) simulated immediate-release (IR) formulations. Intra-cecum (IC) administration simulated large intestine release (LIR) formulations, which provides a time to Cₘₐₓ of more than 4 hours after administration.

Animal studies were performed by Absorption Systems. Test articles: Cysteamine hydrochloride (CH), 98%; Cystamine dihydrochloride (HCCH), 95-96% (both obtained from Sigma Aldrich). CH has one cysteamine base, while HCCH, upon disulfide bond reduction, yields two cysteamine molecules. CH and HCCH were administered at a dose selected to deliver 30 mg/kg of cysteamine base (44.2 mg/kg and 43.8 mg/kg respectively), so that each rat received same number of cysteamine molecules. Test articles (dry powder) were dissolved in 3 mL for PO and in 0.5 mL for IC of room temperature water no more than 15 minutes before dosing.

Five (5) male Sprague Dawley rats ˜300-350 gram (Envigo) were used per group, with 6 groups total. Groups were as shown in Table 2 below:

TABLE 2
Test Groups

| Group | Test Article | Fed Status | Route of Administration |
|---|---|---|---|
| 1 | CH | Fasted | PO |
| 2 | HCCH | Fasted | PO |
| 3 | CH | Fed | PO |
| 4 | HCCH | Fed | PO |
| 5 | CH | Fasted | IC |
| 6 | HCCH | Fasted | IC |

All animals were healthy at the start of the trial and assigned randomly to the study groups by weight. Fed state animals had food and water supplied ad libitum. Fasted state animals had food removed at least 12 hours prior to dosing and food was returned 4 hours post dosing; water was supplied ad libitum. Oral gavage needle and syringe were used for oral administration. For IC dose administration, the animals were anesthetized with isoflurane regulated at ˜1-3% in oxygen. The abdominal cavity was surgically opened and the proximal large intestine was exposed. A test article was administered directly into the proximal large intestine with a 1 cc disposable syringe equipped with a 25 g hypodermic needle. The surgical site was then closed with staples or suture.

Blood was sampled from jugular vein at the following time points: pre-dose, 15, 30, 60, 120, 180, and 240 minutes post dose. Blood samples were collected into tubes containing TBD anticoagulant, then centrifuged at a temperature of 4° C., at 3,000×g, for 5 minutes. Once prepared, plasma samples were snap frozen. Plasma samples were stored at −80° C. until analysis.

Sample analysis was performed by Integrated Analytical Solutions. To measure plasma cysteamine, to 10 uL of a plasma sample or standard was added 10 uL of internal standard with reduction solution: 5 ug/mL d4-cysteamine (CDN Isotopes), 10 mM TCEP in water, prepared fresh, and mixed thoroughly. The mixture was incubated at 37° C. for 60 minutes, then 40 uL of 5% trichloroacetic acid was added and the solution was mixed thoroughly. Samples were then centrifuged at 6100 g for 30 minutes. Next, 20 uL of each supernatant were transferred to an autosampler plate and sealed with a cap mat. The concentration of cysteamine was measured by use of LC/MS/MS tandem mass spectroscopy. Column Atlantis T3 2.1×30mm, 3um was used at a room temperature; mobile phase A: 0.1% heptafluorobutyric acid in water; mobile phase B: 0.2% formic acid in acetonitrile; injection volume: 3 uL. (API 4000 LC/MS/MS; Applied Biosystems). The following MS parameters were used—HPLC system: Shimadzu VP Series 10 System; MS/MS: Applied Biosystems/MDS SCIEX API 4000; software: Analyst v1.6.3; isolation method: Electrospray, Positive Ion; source temperature: TurbolonSpray (ESI) at 400°. Cysteamine concentrations were calculated with a calibration curve that was prepared by spiking plasma with buffered cysteamine solutions, and quality control samples were analyzed with each batch.

### Results

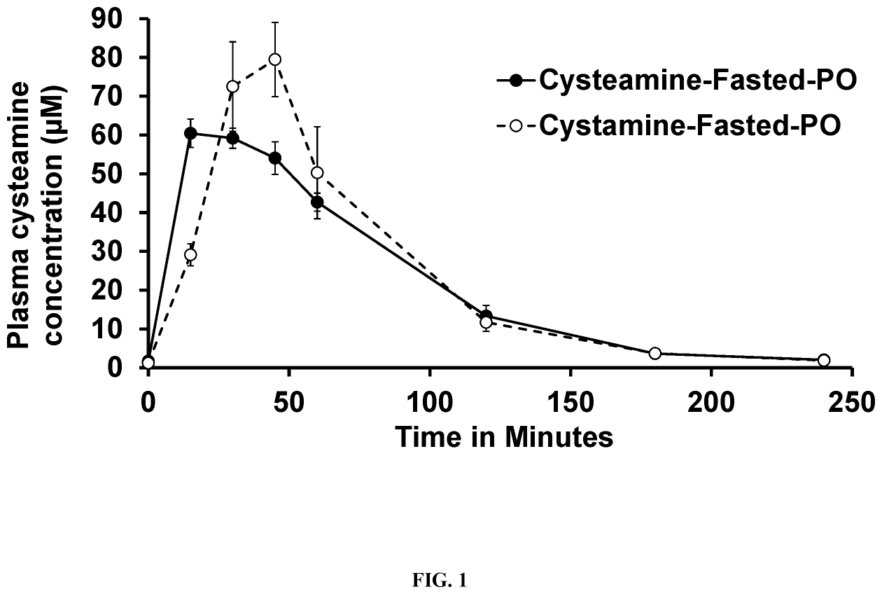

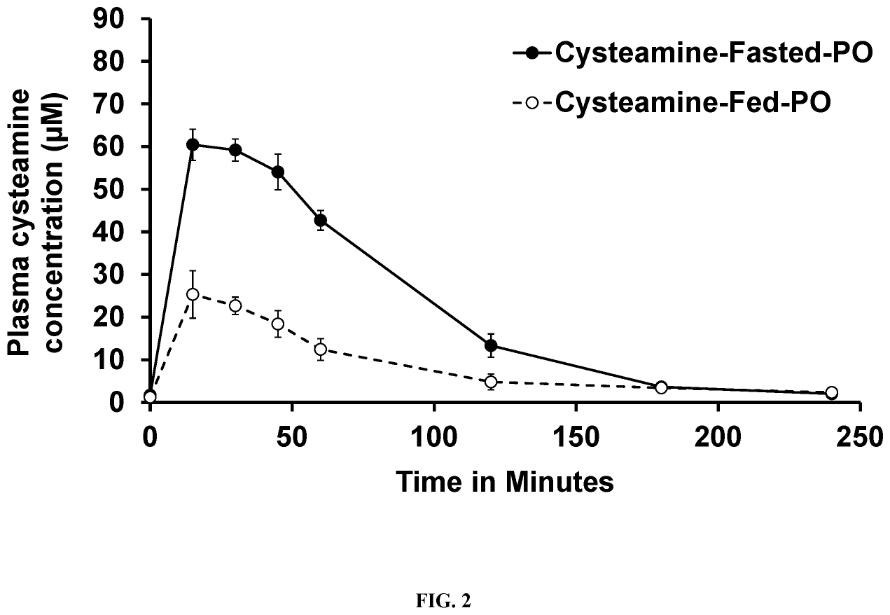

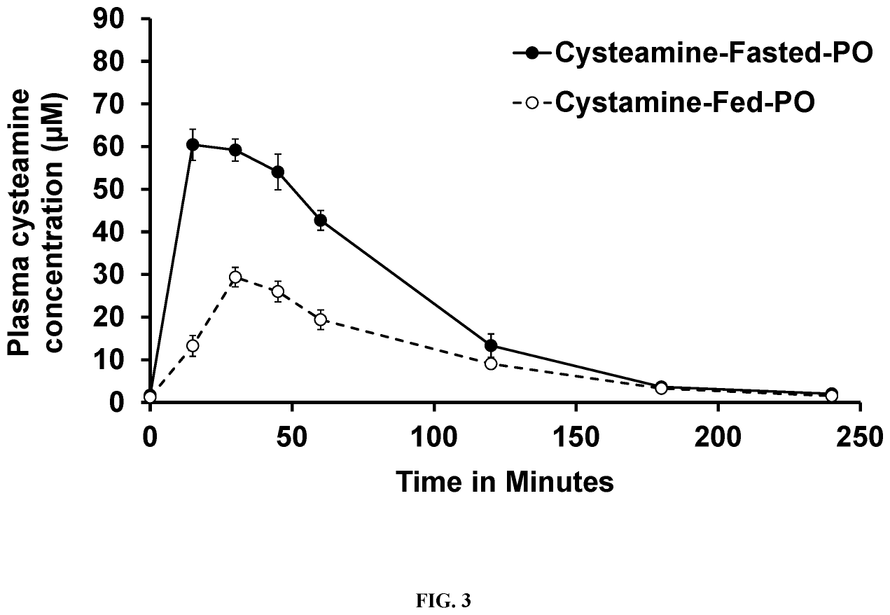

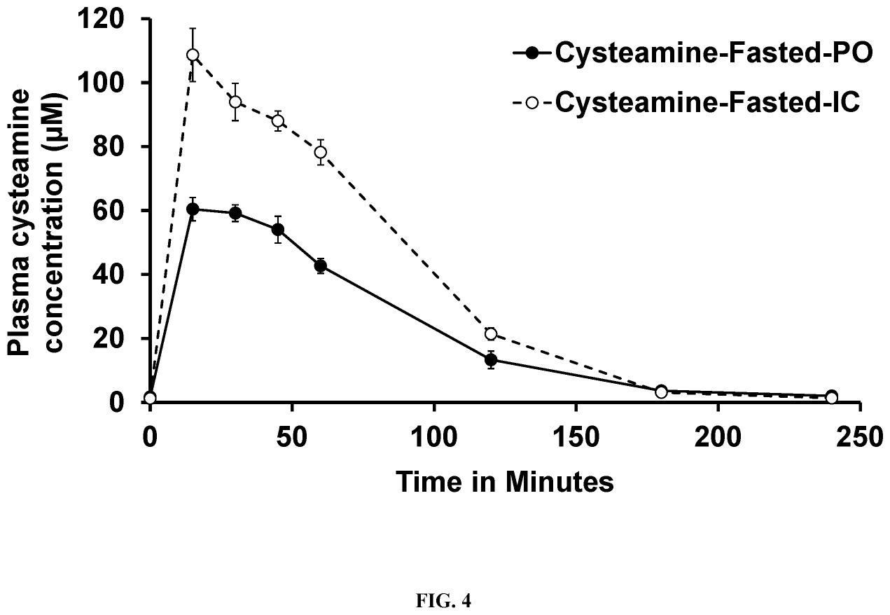

Group 1, cysteamine hydrochloride oral administration to fasted animals represents treatment by Cystagon®, an immediate release of cysteamine bitartrate, which is recommended to be taken on empty stomach. Cystagon® is approved by the FDA for treatment of cystinosis, and hence Group 1 was used as a comparator to all other Groups. FIGS. 1-5 include a curve of Group 1 results. Upon administration of the test articles orally on empty stomach, HCCH showed better absorption as compared to CH, with a higher Cmax (FIG. 1). Area under the curve (AUC), calculated based on trapezoidal rule, was also higher for HCCH (5589) vs CH (5296). Oral administration of the test articles to fed rats revealed reduced absorption for both CH and HCCH (FIG. 2 and FIG. 3, although absorption dropped more for CH (AUC=2032) compared to HCCH (AUC=2550). FIG. 4 shows concentration-time curve for IC administration of CH. Absorption of CH in the large intestine is higher than in the stomach with AUC=8809 vs AUC=5296, respectively. Cmax is also higher, but Tmax (time to Cmax) stayed the same and equal to 15 min (the earliest time blood collection after administration).

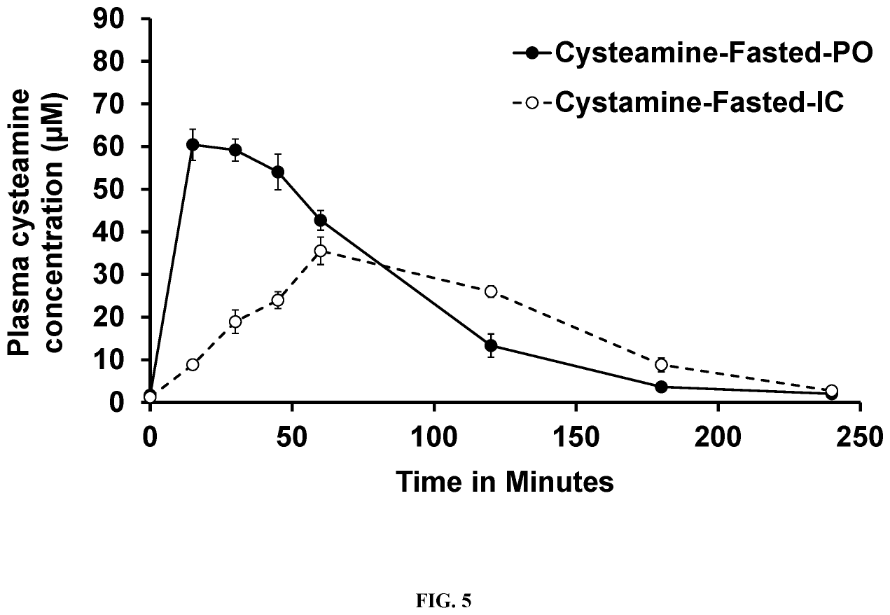

Cystamine dihydrochloride was dosed directly to the large intestine, resulting in its absorption into blood, which was not previously known. It was unexpectedly observed that absorption is not rapid, but rather sustained, without a sharp high blood peak curve of the plasma cysteamine, but rather with a broad curve (FIG. 5). This absorption pattern was unexpected in view of the different pattern of CH absorption dosed in the large intestine, with Cmax at 15 minutes, and a relatively quick decline of cysteamine blood concentration. Similarly, HCCH oral dosing (FIG. 1) showed a sharper peak with a relatively quick decline of cysteamine blood concentration. AUC for HCCH dosing into the large intestine was 4289, which is lower than AUC for oral dosing by 25%. While Cmax for IC dosing of HCCH is more than 50% lower than Cmax after oral administration, it is known that high Cmax is associated with adverse effects. Accordingly, delivering smaller doses of HCCH for immediate release (i.e. stomach release) and higher doses as large intestine release will maintain a Cmax that is lower than the Cmax level of CH, while still with therapeutically sufficient cumulative AUC profile.

I claim:

1. A composition comprising cystamine or a pharmaceutically acceptable salt thereof and one or more pharmaceutically acceptable carriers, diluents, or excipients.

2. The composition of claim 1, wherein the pharmaceutically acceptable salt is cystamine dihydrochloride.

3. The composition of claim 1 or claim 2, wherein the composition is formulated for oral administration.

4. An immediate release (IR) formulation of cystamine or a pharmaceutically acceptable salt thereof comprising the composition of any of claims 1-3, wherein the composition is formulated for immediate release (IR).

5. A large intestine release (LIR) formulation of cystamine or a pharmaceutically acceptable salt thereof comprising the composition of any of claims 1-3, wherein the composition is formulated for large intestine release (LIR).

6. The LIR formulation of cystamine of claim 5, comprising a core comprising cystamine and one or more time-release coatings.

7. The LIR formulation of claim 6, wherein the core comprises a binder.

8. The LIR formulation of claim 7, wherein the core further comprises a filler, a disintegrant, a lubricant, a glidant, or a combination of thereof.

9. The LIR formulation of any of claims 6-8, wherein the one or more time-release coatings are intermediate coatings, functional coatings, or a combination thereof.

10. The LIR formulation of claim 9, wherein the intermediate coating comprises a binder, a salt, or a combination thereof.

11. The LIR formulation of claim 9, wherein the functional coating comprises Eudragit® RL, Eudragit® RS, an odor masking agent, or a combination thereof.

12. A unit dosage form of cystamine or a pharmaceutically acceptable salt thereof comprising one or more of an IR formulation and/or an LIR formulation of cystamine or a pharmaceutically acceptable salt thereof.

13. The unit dosage form of claim 12 comprising in total about 40% of a daily dose of cystamine.

14. The unit dosage form of claim 12 comprising an IR formulation and an LIR formulation, wherein the IR formulation comprises about 10% to about 30% of a daily dose of cystamine and the LIR formulation comprises about 15% to about 35% of a daily dose of cystamine.

15. The unit dosage form of claim 14, wherein the IR formulation comprises 16.6% of a daily dose of cystamine and the LIR formulation comprises about 23.4% of a daily dose of cystamine.

16. A method of treating a subject with a cysteamine or cystamine sensitive disease or disorder, comprising administering to the subject a therapeutically effective amount of the composition, formulation, or unit dosage form of any one of claims 1-15.

17. The method of claim 16, wherein the composition, formulation, or unit dosage form is administered orally.

18. The method of claim 16 or claim 17, wherein the therapeutic level of cysteamine or cystamine is maintained at a constant level.

19. The method of any of claims 16-18, wherein the LIR formulation has a Cₘₐₓ of more than 4 hours after administration.

20. The method any of claims 16-19, wherein the composition, formulation, or unit dosage form is administered 2-3 times per day.

21. The method of any of claims 16-20, wherein the total daily dose of cystamine is about 1.35 g/m² body surface area per day or less.

22. The method of any of claims 16-21, wherein the composition, formulation, or unit dosage form is administered at an amount sufficient to maintain white blood cell cystine levels below 2 nmol half cystine/mg white cell protein.

23. The method of claim 22, wherein the white blood cell cystine levels are maintained below 1 nmol half cystine/mg white cell protein.

24. The method of any of claims 16-23, wherein the cysteamine or cystamine sensitive disease or disorder is cystinosis.

25. A method of treating a subject with a cysteamine or cystamine sensitive disease or disorder, comprising administering to the subject a therapeutically effective amount of the unit dosage form of any one of claims 12-15, wherein the unit dosage form comprises one or more tablets of IR cystamine and one or more tablets of LIR cystamine.

26. A formulation of cystamine comprising:
i) a core comprising cystamine and a binder, and
ii) one or more time-release coatings surrounding the core,
wherein the one or more coatings release the cystamine when the formulation reaches the large intestine, or wherein the formulation has a Cₘₐₓ more than 4 hours when administered therapeutically.

27. A kit comprising:
i) the composition, formulation, or unit dosing form of any one of claims 1-15;
ii) instructions for the administration of the composition, formulation, or unit dosing form for the treatment of a cysteamine or cystamine sensitive disease or disorder.
